(19) United States
(12) Patent Application Publication
(10) Pub. No.: US 2009/0317432 A1
(43) Pub. Date: Dec. 24, 2009

(54) FELT-TIP PEN FOR MAKING UP OR CARING FOR NAILS, SKIN, LIPS EYELASHES OR TEETH

(75) Inventor: Guillaume Kergosien, Chaville (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 12/513,786

(22) PCT Filed: Dec. 13, 2007
(86) PCT No.: PCT/IB07/55398

§ 371 Date: Aug. 18, 2009

Related U.S. Application Data
(60) Provisional application No. 60/885,462, filed on Jan. 18, 2007.

(30) Foreign Application Priority Data
Dec. 14, 2006 (FR) 06 55530

(51) Int. Cl. A61K 8/02; A61Q 3/02; A61Q 1/00; A45D 40/26; B43K 8/06

(52) U.S. Cl. 424/401; 132/200; 132/320; 401/198

(57) ABSTRACT

The present invention relates to an assembly for the application of a nail varnish composition or a composition for making up and/or caring for keratin materials chosen from the skin, the lips and the eyelashes, or the teeth, comprising:
a felt-tip applicator, said applicator being an applicator pen comprising a block of porous material impregnated with the composition, and a wick for transporting the composition by capillary action, and
a nail varnish composition, or a composition for making up and/or caring for keratin materials, with a continuous aqueous phase comprising a particulate phase having an average particle size of less than or equal to 0.5 μm, at least one film-forming polymer and a dispersing agent.

The present invention relates to a felt-tip applicator, in particular a pen, comprising a composition for coating keratin materials such as the skin, the eyelashes, the lips or the nails, and for coating the teeth. They may be compositions for making up and/or caring for keratin materials or the teeth.

In particular, the composition is a nail varnish with a continuous aqueous phase.

Conventionally, making up and/or caring for nails or false nails is carried out using a nail varnish composition applied by means of a brush.

At the current time, the majority of compositions in the form of nail varnishes are based on a mixture of organic solvents containing nitrocellulose, an arylsulphonamide formaldehyde resin or an alkyd resin and a plasticizer. Due to the presence of organic solvents, such varnishes have a certain number of drawbacks associated mainly with the use of solvent and which, in particular, very commonly generate an unpleasant odour when they are applied.

In the last few years, research has therefore been directed towards the development of nail varnishes free of organic solvents, and in particular of aqueous varnishes.

Such aqueous varnishes have been used in application devices other than brushes. For example, document FR 2 537 871 describes a water-based, low-viscosity nail varnish completely free of pigments, containing colouring substances such as acidic organic colouring substances, that can be adapted for application by means of a device equipped with an application wick. However, this type of varnish has the drawback that it impregnates the nails, making it difficult to remove the makeup, which is not desirable. In addition, such makeup may be excessively transparent and lack coverage.

Document EP 170 000 describes, moreover, a nail varnish composition with a low viscosity which comprises pigments and which can be applied by means of an applicator having a substantially non-bristle nib.

Furthermore, document U.S. Pat. No. 6,209,548 describes an applicator for nail paint comprising a reservoir and a nib coupled to said reservoir, and in which the nail paint is essentially water-based and has a viscosity of more than 20 cps but sufficiently low to allow it to flow through the nib.

Document GB 1,193,829 describes a composition for making up the face, comprising finely crushed pigments.

Document EP 1 698 376 describes aqueous varnish compositions.

The invention is therefore directed towards an assembly for application of a nail varnish composition for making up and/or caring for nails which is easy, precise and rapid while at the same time obtaining a film of varnish exhibiting good coverage. In addition, the film obtained exhibits good homogeneity, in particular the colour generated is uniform and the film obtained is uniform, smooth and even. This type of need extends to making up and/or caring for keratin materials chosen from the skin, the lips and eyelashes, or the teeth.

Thus, according to a first aspect, the present invention relates to an assembly for the application of a nail varnish composition or a composition for making up and/or caring for keratin materials chosen from the skin, the lips and the eyelashes, or the teeth, comprising:
a felt-tip applicator, said applicator being an applicator pen comprising a block of porous material impregnated with the composition, and a wick for transporting the composition by capillary action, and
a nail varnish composition, or a composition for making up and/or caring for keratin materials, with a continuous aqueous phase comprising a particulate phase having an average particle size of less than or equal to 1 μm and at least two film-forming polymers.

For this assembly, according to a specific embodiment, the average size of the particles consisting of the particulate colouring substances, the pearlescent agents and/or the fillers is between 0.1 and 1 μm, preferably between 0.1 and 0.7 μm, in particular between 0.1 and 0.5 μm, or even between 0.1 and 0.3 μm.

According to a second aspect, the present invention also proposes an assembly for the application of a nail varnish composition or a composition for making up and/or caring for keratin materials chosen from the skin, the lips and the eyelashes, or the teeth, comprising:
a felt-tip applicator, said applicator being an applicator pen comprising a block of porous material impregnated with the composition, and a wick for transporting the composition by capillary action, and
a nail varnish composition, or a composition for making up and/or caring for keratin materials, with a continuous aqueous phase comprising a particulate phase having an average particle size of less than or equal to 0.5 μm, at least one film-forming polymer and a dispersing agent.

For this assembly, according to a specific embodiment, the average size of the particles consisting of the particulate colouring substances, the pearlescent agents and/or the fillers is between 0.1 and 0.5 μm, or even between 0.1 and 0.3 μm.

In particular, an advantage of the assembly proposed in the present application is that several successive layers can be readily applied. Thus, the layers applied neither become detached nor degrade during the application of the additional layers. In other words, previous layers are preserved during the application of successive layers.

For the purpose of the present invention, the term “composition with a continuous aqueous phase” is intended to mean that the composition has a conductivity, measured at 25° C., of greater than or equal to 23 μS/cm (microSiemens/cm), the conductivity being measured, for example, using a Mettler Toledo MPC227 conductimeter and an Inlab730 conductivity measuring cell. The measuring cell is immersed in the composition so as to eliminate the air bubbles that may form between the 2 electrodes of the cell. The conductivity is read as soon as the value of the conductimeter is stabilized. A mean is calculated over at least 3 successive measurements.

For the purpose of the present invention, the term “pen” denotes an instrument that can be gripped, consisting of a tube ending with a tip and containing a reservoir of a liquid that flows out through the tip as soon as it is applied to a support.

The term “felt-tip” denotes a tip of felt or of synthetic fibres.

In particular, in the context of the present invention, the term “felt-tip pen” is different from a ball-point pen or from any device based on single bristles used for the application of conventional nail varnish compositions. Finally, the method of application by the applicator considered in the present application is capillary action. Thus, the present application is not intended to cover assemblies in which the applicator is equipped with a valve.

Compositions

Particle Size

The compositions according to the present invention comprise a particulate phase. This particulate phase is characterized by a given average particle size.

Such a particle size can be measured by various techniques. Mention may in particular be made of (dynamic and static) light scattering techniques, Coulter capture methods, measurements of sedimentation rate (related to the size via Stokes law) and microscopy.

These techniques make it possible measure to a particle diameter and, for some particles, a particle size distribution.

Preferably, the sizes and the size distributions of the particles of the compositions according to the invention are measured by static light scattering using a commercial particle sizer of MasterSizer 2000 type from Malvern. The data are processed on the basis of the Mie diffusion theory. This theory, which is true for isotropic particles, makes it possible to determine, in the case of nonspherical particles, an “effective” particle diameter. This theory is in particular described in the book by Van de Hulst, H. C., “Light Scattering by Small Particles”, Chapters 9 and 10, Wiley, New York, 1957.

In the context of the present invention, the “average particle size” is expressed as volume-average “effective” diameter D[4,3], defined in the following way:

$$D [ 4 , 3 ] = \frac{{\sum }_{i} {V}_{i} \cdot {d}_{i}}{{\sum }_{i} {V}_{i}}$$

where Vᵢ represents the volume of the particles of effective diameter dᵢ. This parameter is in particular described in the technical documentation of the particle sizer.

The measurements are carried out at 25° C., on a dilute dispersion of particles. The “effective” diameter is obtained by taking a refractive index of 1.33 for water and an average refractive index of 2.52 for the particles. The blue obscuration is removed.

Thus, according to the invention, the particles of the particulate phase of the compositions in accordance with the invention have an average size, expressed as volume-average “effective” diameter D[4,3], of less than or equal to 1 μm, preferably less than or equal to 0.7 μm, or even 0.5 μm, for example less than or equal to 0.3 μm. As regards the particulate colouring substances, the pearlescent agents and/or the fillers as detailed hereinafter, the average size is preferably between 0.1 and 1 μm, preferably between 0.1 and 0.7 μm, and even more preferably between 0.1 and 0.5 μm, or even between 0.1 and 0.3 μm.

This average particle size is particularly advantageous in terms of the use of the composition in accordance with the present invention compared with compositions comprising particles of larger average size which may in particular require large amounts of conventional thickener, so as to prevent their sedimentation, and resulting in an increase in the viscosity of the composition which is prejudicial to good spreading of the composition.

### PARTICULATE PHASE

According to a specific embodiment of the invention, the particulate phase comprises, or even consists mainly of, particulate colouring substances such as pigments needed in particular to give a coloured appearance to the compositions that can be used in particular for making up nails, but also for making up and/or caring for keratin materials chosen from the skin, the lips and the eyelashes, and the teeth.

However, the particulate phase may also contain other types of particles, such as pearlescent agents or fillers.

When, according to a specific embodiment, the film-forming polymer is in the dispersed state, in particular in the form of a latex, the polymer particles of which it is made up are also part of the composition of the particulate phase. As is subsequently disclosed in the present description, the size of the polymer particles in this specific embodiment is less than 1 μm, or even less than 0.5 μm.

The term “pigments” should be understood to mean white or coloured, mineral or organic particles of any shape, that are insoluble in the physiological medium and are intended to confer a tint on the composition.

The term “pearlescent agents or pearlescent pigments” should be understood to mean iridescent particles of any shape, in particular produced by certain mollusks in their shell, or else synthesized and which produce a colour effect by optical interference.

The pigments may be white or coloured, and mineral and/or organic. Among mineral pigments, mention may be made of titanium dioxide, optionally surface-treated, zirconium or cerium oxides, and also zinc oxides, iron (black, yellow or red) oxides or chromium oxides, manganese violet, ultramarine blue, chromium hydrate and ferric blue, and metal powders such as aluminium powder and copper powder.

Among organic pigments, mention may be made of carbon black, pigments of D & C type, and lakes based on cochineal carmine, on barium, on strontium, on calcium and on aluminium.

The pearlescent agents may be chosen from pearlescent pigments such as mica coated with titanium or with bismuth oxychloride, coloured pearlescent pigments such as titanium mica coated with metal oxides such as iron oxide or chromium oxide, titanium mica coated with an organic pigment of the abovementioned type, and also pearlescent pigments based on bismuth oxychloride.

Mention may also be made of pigments with effects, such as:
particles with a metal tint, chosen from particles of at least one metal and/or of at least one metal derivative or particles comprising an organic or mineral, single-material or multi-material substrate at least partially coated with at least one layer with a metal tint comprising at least one metal and/or at least one metal derivative,
reflective pigments,
goniochromatic colouring agents, and
mixtures thereof.

These pigments with effects, in particular the particles with a metal tint, may be in the form of flakes.

The pigments and pearlescent agents may be present in the composition in a proportion of from 0.01% to 15% by weight, in particular from 0.01% to 10% by weight, and in particular from 0.02% to 5% by weight.

The term “fillers” should be understood to mean colourless or white, mineral or synthetic particles of any shape that are insoluble in the medium of the composition.

By way of fillers, mention may in particular be made of talc, zinc stearate, mica, kaolin, polyamide (Nylon®) powders (Orgasol® from Arkema), polyethylene powders, tetrafluoroethylene (Teflon®) polymer powders, starch, boron nitride, polymeric microspheres such as those of polyvinylidene chloride/acrylonitrile, such as Expancel® (Nobel Industrie), or those of acrylic acid copolymers (Polytrap® from the company Dow Corning) and silicone resin microbeads (Tospearls® from Toshiba, for example), elastomeric organopolysiloxanes, precipitated calcium carbonate, magnesium carbonate, magnesium hydrogen carbonate, hydroxyapatite, hollow silica microspheres (Silica Beads® from Maprecos), glass or ceramic microcapsules, metal soaps derived from organic carboxylic acids containing from 8 to 22 carbon atoms, preferably from 12 to 18 carbon atoms, for example zinc stearate, magnesium stearate or lithium stearate, zinc laurate or magnesium myristate.

The particulate phase consisting of the particulate colouring substances may be present in the compositions in accordance with the invention at a content ranging from 0.01% to 20% by weight, especially from 0.05% to 15% by weight, and in particular from 0.1% to 10% by weight, relative to the total weight of the composition.

According to another specific embodiment of the invention, when the film-forming polymer is in the form of particles in an aqueous dispersion, the overall solids content, which thus comprises the particulate phase including the particles of film-forming polymer, is less than 50%, preferably between 20% and 40%, preferably between 25% and 35%. This in particular allows good coverage and/or good hold of the composition.

According to a specific embodiment of the invention, the particulate phase comprises pigments whose size is between 0.1 and 1 μm, preferably between 0.1 and 0.7 μm, and even more preferably between 0.1 and 0.5 μm, or even between 0.1 and 0.3 μm, and a film-forming polymer in the disperse state, in which the particles of polymer of which it is made up have a size of between 1 nm and 1 μm, in particular between 5 and 500 nm, and more particularly between 10 and 250 nm. According to yet another embodiment of the present invention, the particulate phase comprises pigments whose size is between 0.1 and 0.3 μm, and a film-forming polymer in the disperse state, in which the particles of polymer of which it is made up have a size of between 10 and 250 nm.

Dispersing Agent

The dispersing agent may consist of a mixture of dispersing agents.

The dispersing agent may belong to various chemical families detailed hereinafter:

1/Ionic surfactants such as:
carboxylic salts,
salts of amino acids such as glutamic acid,
salts of sulphosuccinic acids,
salts of phosphoric acid.

The term “salts” is intended to mean the alkali metal, such as Na, Li, K, salts of said acids, the mono-, di- or triethanolamine salts or the basic amino acids such as lysine or arginine, of said acids, and mixtures thereof;
oxyethylenated derivatives of phosphoric acid, and in particular:
   esters derived from the reaction of phosphoric acid and of at least one alcohol comprising a saturated or unsaturated, linear or branched alkyl chain containing from 8 to 22 carbon atoms,
   esters derived from the reaction of phosphoric acid and of at least one ethoxylated alcohol comprising a saturated or unsaturated, linear or branched alkyl chain containing from 8 to 22 carbon atoms and comprising from 2 to 40 oxyethylenated groups,
   salts thereof,
   and mixtures thereof.

These esters may in particular be chosen from esters of phosphoric acid and of C₉-C₁₅ alcohols or their salts, such as the potassium salt of (C₉-C₁₅)alkyl phosphate sold under the name Arlatone MAP by the company Uniqema, esters of phosphoric acid and of stearyl and/or isostearyl alcohols, such as the stearyl/isostearyl alcohol phosphate (CTFA name: Octyldecylphosphate) sold under the name Hostaphat CG120 by the company Hoechst Celanese, esters of phosphoric acid and of cetyl alcohol, and their oxyethylenated derivatives, such as the product sold under the name Crodafos CES (mixture of cetearyl alcohol, of dicetyl phosphate and of ceteth phosphate) by the company Croda, esters of phosphoric acid and of tridecyl alcohol, and their oxyethylenated derivatives, such as the product sold under the name Crodafos T10 (CTFA name: Trideceth-10 Phosphate) by the company Croda;
alkyl sulphates,
alkyl benzenesulphonates,
alkyl ether citrates that may be chosen in particular
   from the group comprising monoesters, diesters or triesters derived from the reaction of citric acid and of at least one oxyethylenated fatty alcohol comprising a saturated or unsaturated, linear or branched alkyl chain containing from 8 to 22 carbon atoms and comprising from 3 to 9 oxyethylenated groups, and mixtures thereof.

Mention may, for example, be made of mono-, di- and triesters of citric acid and of ethoxylated lauryl alcohol, comprising from 3 to 9 oxyethylenated groups, sold by the company Witco under the name Witconol EC, in particular Witconol EC 2129, which is predominantly a dilaureth-9 citrate, and Witconol EC 3129, which is predominantly a trilaureth-5,9 citrate.

The alkyl ether citrates may advantageously be used in a form neutralized at approximately pH 7, the neutralizing agent being chosen from inorganic bases such as sodium hydroxide, potassium hydroxide or ammonia, and organic bases such as mono-, di- and triethanolamine, aminomethyl-1,3-propanediol, N-methylglucamine, basic amino acids such as arginine and lysine, and mixtures thereof.

2/Nonionic surfactants such as:
ethers of C₁₀-C₃₀ fatty alcohols and of polyethylene glycol (also known as polyoxyethylene alkyl ethers, comprising from 2 to 100 oxyethylenated groups, such as those sold under the name Brij by the company Uniqema,
esters of a fatty acid (in particular of a C₈-C₂₄, preferably C₁₆-C₂₂, acid) and of polyethylene glycol (also known as polyoxyethylene alkyl esters), comprising from 2 to 100 oxyethylenated groups, such as those sold under the name Myrj by the company Uniqema,
sorbitan alkyl esters such as those sold under the name Span by the company Uniqema,
polyoxyethylene sorbitan alkyl esters comprising from 4 to 100 oxyethylenated groups (such as those sold under the name Tween by the company Uniqema),
esters of C₈-C₂₄ fatty acids and of polyglycerol (in particular tetraglycerol, hexaglycerol or decaglycerol), comprising from 2 to 10 oxyethylenated groups (such as those sold under the name Tetraglyn, Hexaglyn, Decaglyn by the company Nikkol),
polyhydroxystearic acid esters (such as, for example, Arlacel 165 from the company Uniqema),
alkyl glucosides, for instance the compounds derived from the reaction of coconut alcohol and of glucose (INCI name: coco-glucosides), such as those sold under the reference APG by the company Cognis), and alkyl maltosides.

Mention may also be made of silicone surfactants, in particular oxyalkylenated silicones, also known as dimethicone copolyols.

The dimethicone copolyols in accordance with the invention are more preferably chosen from the compounds of general formula (I):

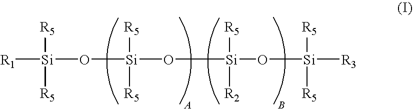

in which:
R₁, R₂ and R₃, independently of one another, represent an alkyl, aryl or aralkyl group containing not more than 10 carbon atoms, or a radical —(CH₂)ₓ—(OCH₂CH₂)_{y}(OCH₂—CH₂—CH₂)_{z}—OR₄, at least one R₁, R₂ or R₃ radical not being an alkyl radical, R₄ being a hydrogen, an alkyl radical or an acyl radical; some of the R₁, R₂ and R₃ radicals may also contain, in addition, an ethyl cyclohexylene monooxide group and are present in the polysiloxane chain in a low proportion.
The R₅ radicals, which may be identical or different, represent an alkyl, aryl or aralkyl group containing not more than 10 carbon atoms, and preferably chosen from C₁-C₄ lower alkyls such as methyl, ethyl or butyl, or else chosen from phenyl and benzyl groups, and even more preferably all denote methyl groups; some of the R₅ radicals may also contain, in addition, an ethylcyclohexylene monooxide group and are present in the polysiloxane chain in a low proportion.
A is an integer ranging from 0 to 200; B is an integer ranging from 0 to 50; on the condition that A and B are not equal to zero at the same time; x is an integer ranging from 1 to 8; y is an integer ranging from 1 to 30; z is an integer ranging from 0 to 5.

According to a preferred embodiment of the invention, in the compound of formula (I), the alkyl radical is a methyl radical, x is an integer ranging from 2 to 6 and y is an integer ranging from 4 to 30. By way of example of silicone surfactants of formula (I), mention may be made of the compounds of formula (II):

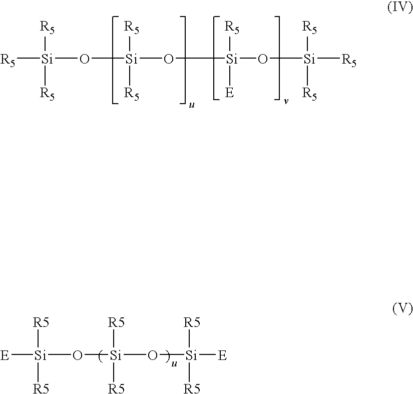

in which A is an integer ranging from 20 to 105, B is an integer ranging from 2 to 10 and y is an integer ranging from 10 to 20. By way of example of silicone surfactants of formula (I), mention may also be made of the compounds of formula (III):

HO—(OCH₂CH₂)_{y}—(CH₂)₃—[(CH₃)₂SiO]_{A′}—(CH₂)₃—(OCH₂CH₂)_{y}—OH (III)

in which A′ and y are integers ranging from 10 to 20.

As compounds of the invention, those sold by the company Dow Corning under the names DC 5329, DC 7439-146, DC2-5695 and Q43667 may be used.

The compounds DC 5329, DC 7439-146 and DC2-5695 are compounds of formula (II) in which, respectively, A is 22, B is 2 and y is 12; A is 103, B is 10 and y is 12; A is 27, B is 3 and y is 12. The compound Q4-3667 is a compound of formula (III) in which A is 15 and y is 13.

According to another preferred embodiment of the invention, by way of example of silicone surfactants of formula (I), mention may be made of the following formulae (IV) and (V):

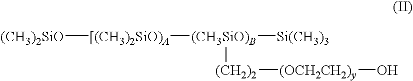

in which:
u is 5 to 59, preferably 10 to 50, and more preferably 12 to 25;
v is 3 to 12, preferably 4 to 10, and more preferably 5 to 8;
E denotes a group —(CH₂)— (OCH₂CH₂)_{y}(OCH₂—CH₂—CH₂)—OR₄ in which:
   x is 1 to 8, preferably from 2 to 4, and more preferably 3;
   y>0 and z≧0; y and z are chosen such that the total molar mass of the radical E ranges from 200 to 10 000 g/mol, and more preferably from 350 to 3000; preferably, the number z is equal to zero.

In the formula E, when z is positive, the oxyethylene and oxypropylene units may be distributed randomly in the polyether chain E, and/or in the form of blocks.

The water-soluble silicones in accordance with the invention are known and in particular described in U.S. Pat. No. 5,338,352, and the method for preparing them is described in particular in U.S. Pat. No. 4,847,398.

Such silicones are, for example, sold by the company OSI under the trade names Silwet L-720®, Silwet L-7002®, Silwet L-7600®, Silwet L-7604®, Silwet L-7605®, Silwet L-7607®, Silwet 1614, Silwet L-7657®, Silwet L-7200®, Silwet L7230, Silsoft 305, Silsoft 820 and Silsoft 880, or else by the company Goldschmidt under the trade names Tegowet 260, Tegowet 500, Tegowet 505 and Tegowet 510.

3/Ionic polymers such as:
polyacrylic acids and salts thereof,
styrene/acrylic acid copolymers and salts thereof,
vinylnaphthalene/acrylic acid copolymers and salts thereof,
styrene/maleic acid copolymers and salts thereof,
vinylnaphthalene/maleic acid copolymers and salts thereof,
copolymers of maleic anhydride and of diisobutylene, and salts thereof, and more particularly the copolymer sold under the name Orotan 731DP® by the company Rhodia,
acrylic acid copolymers and salts thereof,
maleic anhydride copolymers and salts thereof, in particular the copolymers obtained by copolymerization of one or more maleic anhydride comonomers and of one or more comonomers chosen from vinyl acetate, vinyl alcohol, vinylpyrrolidone, olefins containing from 2 to 20 carbon atoms such as octadecene, ethylene, isobutylene, diisobutylene, isooctylene and styrene, and mixtures thereof, it being possible for the maleic anhydride comonomers to be optionally partially or completely hydrolysed.

Mention may, for example, be made of the styrene/maleic anhydride (50/50) copolymer, in the form of an ammonium salt at 30% in water, sold under the reference SMA1000H® by the company Arkema, or the styrene/maleic anhydride (50/50) copolymer, in the form of a sodium salt at 40% in water, sold under the reference SMA1000HNa® by the company Arkema;
noncrosslinked N-vinylimidazole polymers or copolymers (the term “noncrosslinked” is intended to mean any polymer comprising N-vinylimidazole units and not comprising a crosslinking agent), in particular the copolymers combining N-vinylimidazole subunits with N-vinylpyrrolidone and/or vinylcaprolactam subunits.

To this effect, use may be made of the vinyl-pyrrolidone/vinylimidazole (50/50) copolymer having a weight-average molar mass of 1 200 000, sold under the reference Luvitec VPI 55K72W by the company BASF or the vinylpyrrolidone/vinylimidazole (50/50) copolymer having a weight-average molar mass of 10 000, sold under the reference Luvitec VPI 55K18P by the company BASF;
polymers obtained by copolymerization or grafting of hydrophobic groups with the ionic monomers mentioned above,
2-acrylamido-2-methylpropanesulphonic acid (AMPS) polymers and copolymers, such as, for example, Aristoflex sold by the company Clariant,
water-dispersible polymers of isophthalic acid or of sulphoisophthalic acid, and in particular the phthalate/sulphoisophthalate/glycol copolymers (such as, for example, diethylene glycol/phthalate/isophthalate/1,4-cyclohexanedimethanol), sold under the name “Eastman AQ polymer” (in particular AQ35S, AQ38S, AQ55S, AQ48 Ultra) by the company Eastman Chemical,
and blends thereof.

According to a specific embodiment of the invention, the dispersing agent is a polyacrylic acid in the form of a salt or a styrene/acrylic acid copolymer in the form of a salt.

4/Nonionic polymers such as:
polyvinyl alcohols,
vinylpyrrolidone homopolymers, such as Luviskol K 17 powder, Kollidon 17 PF, Kollidon 12 PF from BASF, Kollidon 30, Kollidon 90 from BASF, polyvinylpyrrolidone K 60 solution from Fluka,
polyalkylene glycols, preferably chosen from those in which the alkylene group contains from 1 to 4 carbon atoms, in particular polyethylene glycols, polypropylene glycols and polybutylene glycols,
block copolymers of ethylene oxide and of propylene oxide, that may be chosen in particular from the block copolymers of formula (VI):

HO(C₂H₄O)ₒ(C₃H₆O)ₚ(C₂H₄O)_{q}H (VI)

in which o, p and q are integers such that o+q ranges from 2 to 100 and p ranges from 14 to 60, and blends thereof, and more particularly from the block copolymers of formula (VI) having an HLB ranging from 2 to 16.

These block copolymers may in particular be chosen from poloxamers (INCI name), and in particular from Poloxamer 231, such as the product sold by the company BASF under the name Pluronic L81, of formula (VI) with o=q=6, p=39 (HLB 2); Poloxamer 282 such as the product sold by the company BASF under the name Pluronic L92, of formula (VI) with o=q=10, p=47 (HLB 6); and Poloxamer 124 such as the product sold by the company BASF under the name Pluronic L44, of formula (VI) with 0=q=11, p=21 (HLB 16).

5/As dispersing agent, mention may also be made of citric acid, alpha-hydroxy acids, and mixtures thereof.

Among all the dispersing agents mentioned above, preference is given to those chosen from anionic polymers such as polyacrylic acid salts, styrene/acrylic acid copolymer salts, vinylnaphthalene/acrylic acid copolymer salts, styrene/maleic acid copolymer salts, vinylnaphthalene/maleic acid copolymer salts, copolymers of maleic anhydride and of diisobutylene, and salts thereof, and more generally all the salts of copolymers of acrylic acid or of maleic anhydride.

According to a specific embodiment of the invention, the dispersing agent is a styrene/acrylic acid copolymer salt.

For obvious reasons, the concentration of dispersing agent is capable of varying significantly from the point of view of, firstly, its chemical nature and, secondly, the size and the chemical nature of the particles with respect to which it must precisely perform this dispersing function.

The dispersing agent may be present at a content ranging from 0.01% to 10% by weight, in particular ranging from 0.1% to 7.5% by weight, or even from 0.1% to 5% by weight, relative to the total weight of the composition in accordance with the invention.

According to a specific embodiment, the dispersing agent is placed at the surface of the pigment particles.

To do this, the pigment particles may advantageously be surface-treated. For example, they may be treated so as to comprise organic functions capable of promoting the adsorption of the dispersing agent. According to a specific embodiment, the pigment particles comprise, after such a treatment, polar functions.

Viscosity

According to a specific embodiment, the compositions in accordance with the present invention have a low viscosity. In particular, the viscosity is advantageously less than or equal to 200 cps. Typically, the viscosity of the composition may range from 2 to 200 cps, preferably from 5 to 150 cps.

The viscosity is, for example, measured using a Brookfield RV apparatus. The protocol for measuring the viscosity is the following. The spindle used is No. 5. The spindle rotation speed is 100 rpm. The measurement is carried out after 10 minutes of rotation. The liquid is placed in a 30 ml flask. The measurement is carried out at a water bath temperature of 25° C.

Such a viscosity makes it possible in particular to obtain ready application of a nail varnish by means of a felt-tip pen.

Film-Forming Polymer

The composition according to the invention comprises one or more film-forming polymers.

In the present application, the term “film-forming polymer” is intended to mean a polymer capable of forming, on its own or in the presence of an auxiliary film-forming agent, a continuous film on a support, at a temperature ranging from 20° C. to 150° C.

According to one embodiment, the composition according to the invention is a nail varnish and comprises at least two film-forming polymers.

In the context of the present invention, it is understood that the two film-forming polymers may be separated or in the form of multiphase particles, as is detailed hereinafter. In the latter case, one polymer may constitute or be included in the core or inner part of the particles and another polymer may constitute or be included in the envelope or shell or outer part of said particles.

According to another embodiment, the composition according to the invention is a composition for coating keratin materials chosen from the skin, the eyelashes and the lips, or the teeth, and comprises at least one film-forming polymer.

According to an advantageous embodiment, the film-forming polymer is present in the form of particles in an aqueous dispersion, generally known as latex or pseudolatex. The techniques for preparing these dispersions are well known to those skilled in the art.

A dispersion suitable for the invention may comprise one or more types of particles, it being possible for these particles to vary by virtue of their size, their structure and/or their chemical nature. In general, these polymer particles have an average size, expressed as volume-average “effective” diameter D[4,3], of less than or equal to 1 μm.

The size of the polymer particles in an aqueous dispersion can range from 1 nm to 1 μm, preferably from 5 to 500 nm, and is in particular from 10 to 250 nm. The size of the polymer particles can be measured by the same method(s) described above for the particulate phase.

These solid particles may be anionic, cationic or neutral in nature and may constitute a mixture of solid particles of different natures.

In the present invention, the term “aqueous” is intended to mean a liquid medium based on water and/or on hydrophilic solvents. This aqueous liquid medium may consist essentially of water. It may also comprise a mixture of water and of water-miscible organic solvent(s) (miscibility in water of greater than 50% by weight at 25° C.), for instance lower monoalcohols containing from 1 to 5 carbon atoms, such as ethanol or isopropanol, glycols containing from 2 to 8 carbon atoms, such as propylene glycol, ethylene glycol, 1,3-butylene glycol or dipropylene glycol, C₃-C₄ ketones and C₂-C₄ aldehydes.

In general, the nail varnish composition or composition for making up and/or caring for keratin materials in accordance with the invention comprises from 0.1% to 60% by weight, and in particular from 1% to 50%, and better still from 5% to 40% by total weight of solids of film-forming polymer, relative to the total weight of the composition.

Among the film-forming polymers that can be used in the composition of the present invention, mention may be made of synthetic polymers, of radical type or of polycondensate type, polymers of natural origin, and blends thereof. In general, these polymers may be random polymers, block copolymers of A-B type, multiblock copolymers A-B-A or alternatively. ABCD, etc., or even grafted polymers.

Radical Film-Forming Polymer

The term “radical polymer” is intended to mean a polymer obtained by a polymerization of unsaturated, in particular ethylenically unsaturated, monomers, each monomer being capable of homopolymerizing (unlike polycondensates).

The radical-type film-forming polymers may in particular be acrylic and/or vinyl homopolymers or copolymers.

The vinyl film-forming polymers may result from the polymerization of ethylenically unsaturated monomers having at least one acidic group and/or esters of these acidic monomers and/or amides of these acidic monomers.

As ethylenically unsaturated monomers having at least one acidic group or monomer bearing an acidic group, use may be made of α,β-ethylenically unsaturated carboxylic acids such as acrylic acid, methacrylic acid, crotonic acid, maleic acid or itaconic acid. Use is in particular made of (meth)acrylic acid and crotonic acid, and more particularly (meth)acrylic acid.

The esters of acidic monomers are advantageously chosen from esters of (meth)acrylic acid (also known as (meth)acrylates), especially alkyl, in particular C₁-C₂₀ alkyl, more particularly C₁-C₈ alkyl(meth)acrylates, aryl, in particular C₆-C₁₀ aryl(meth)acrylates, and hydroxyalkyl, in particular C₂-C₆ hydroxyalkyl(meth)acrylates.

Among the alkyl(meth)acrylates, mention may be made of methyl methacrylate, ethyl methacrylate, butyl methacrylate, isobutyl methacrylate, 2-ethylhexyl methacrylate and lauryl methacrylate.

Among the hydroxyalkyl(meth)acrylates, mention may be made of hydroxyethyl acrylate, 2-hydroxypropyl acrylate, hydroxyethyl methacrylate and 2-hydroxypropyl methacrylate.

Among the aryl(meth)acrylates, mention may be made of benzyl acrylate and phenyl acrylate.

The (meth)acrylic acid esters are in particular alkyl (meth)acrylates.

According to the present invention, the alkyl group of the esters may be either fluorinated or perfluorinated, i.e. some or all of the hydrogen atoms of the alkyl group are substituted with fluorine atoms.

As amides of the acidic monomers, mention may, for example, be made of (meth)acrylamides, and especially N-alkyl(meth)acrylamides, in particular N—(C₂-C₁₂)-alkyl (meth) acrylamides. Among the N-alkyl(meth) acrylamides, mention may be made of N-ethylacrylamide, N-t-butylacrylamide and N-t-octylacrylamide.

The vinyl film-forming polymers may also result from the homopolymerization or copolymerization of monomers chosen from vinyl esters and styrene monomers. In particular, these monomers may be polymerized with acidic monomers and/or esters thereof and/or amides thereof, such as those mentioned above.

As an example of vinyl esters, mention may be made of vinyl acetate, vinyl neodecanoate, vinyl pivalate, vinyl benzoate and vinyl t-butyl benzoate.

As styrene monomers, mention may be made of alpha-methylstyrene styrene.

The list of monomers given is not limiting and it is possible to use any monomer known to those skilled in the art which comes under the categories of acrylic and vinyl monomers (including monomers modified with a silicone chain).

Silicone acrylic polymers may also be used as vinyl polymer.

Mention may also be made of the polymers resulting from the radical polymerization of one or more radical monomers, within and/or partially at the surface of pre-existing particles of at least one polymer chosen from the group consisting of polyurethanes, polyureas, polyesters, polyesteramides and/or alkyds. These polymers are generally referred to as “hybrid polymers”.

Polycondensate

As film-forming polymer of polycondensate type, mention may be made of anionic, cationic, nonionic or amphoteric polyurethanes, polyurethane-acrylics, polyurethane-polyvinylpyrrolidones, polyester-polyurethanes, polyether-polyurethanes, polyureas, polyurea/polyurethanes, silicone polyurethanes, and mixtures thereof.

The film-forming polyurethane may, for example, be an aliphatic, cycloaliphatic or aromatic polyurethane, polyurea/urethane or polyurea copolymer, comprising, alone or as a mixture, at least one block chosen from:
a block of aliphatic and/or cycloaliphatic and/or aromatic polyester origin and/or
a branched or nonbranched silicone block, for example polydimethylsiloxane or polymethylphenylsiloxane, and/or a block comprising fluoro groups.

The film-forming polyurethanes as defined in the invention may also be obtained from branched or nonbranched polyesters, or from alkyds comprising labile hydrogens that are modified by reaction with a diisocyanate and a difunctional (for example dihydro, diamino or hydroxyamino) organic compound, also comprising either a carboxylic acid or carboxylate group, or a sulphonic acid or sulphonate group, or alternatively a neutralizable tertiary amine group or a quaternary ammonium group.

Among the film-forming polycondensates, mention may also be made of polyesters, polyesteramides, fatty-chain polyesters, polyamides and epoxyester resins.

The polyesters may be obtained, in a known manner, by polycondensation of dicarboxylic acids with polyols, in particular diols.

The dicarboxylic acid may be aliphatic, alicyclic or aromatic. As an example of such acids, mention may be made of: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarbocyclic acid, 1,4-cyclohexane-dicarboxylic acid, isophthalic acid, terephthalic acid, 2,5-norboranedicarboxylic acid, diglycolic acid, thiodipropionic acid, 2,5-naphthalenedicarboxylic acid and 2,6-naphthalenedicarboxylic acid. These dicarboxylic acid monomers may be used alone or in combination with at least two dicarboxylic acid monomers. Among these monomers, phthalic acid, isophthalic acid and terephthalic acid are in particular chosen.

The diol may be chosen from aliphatic, alicyclic and aromatic diols. Use is in particular made of a diol chosen from: ethylene glycol, diethylene glycol, triethylene glycol, 1,3-propanediol, cyclohexanedimethanol and 4-butanediol. As other polyols, use may be made of glycerol, pentaerythritol, sorbitol and trimethylolpropane.

The polyesteramides may be obtained in a manner similar to the polyesters, by polycondensation of diacids with diamines or amino alcohols. Ethylenediamine, hexamethylenediamine, meta-phenylenediamine or para-phenylenediamine may be used as diamine. Monoethanolamine may be used as amino alcohol.

Polymer of Natural Origin

In the present invention, use may be made of optionally modified polymers of natural origin, such as shellac resin, sandarac gum, dammar resins, elemi gums, copal resins, water-insoluble cellulose polymers such as nitrocellulose, modified cellulose esters, including in particular carboxyalkylcellulose esters such as those described in Patent Application US 2003/185774, and mixtures thereof.

According to a specific embodiment of the invention, the film-forming polymer(s) is (are) in the dispersed state, chosen from acrylic dispersions, aqueous dispersions of polyurethane, sulphopolyesters, vinyl dispersions, aqueous dispersions of polyvinyl acetate, aqueous dispersions of terpolymer of vinylpyrrolidone/dimethylaminopropylmethacrylamide-lauryldimethylpropylmethacrylamidoammonium chloride terpolymer, aqueous dispersions of polyurethane/polyacrylic hybrid polymers, dispersions of particles of core-shell type, and mixtures thereof.

Various types of aqueous dispersions, in particular commercially available aqueous dispersions, suitable for the preparation of the composition in accordance with the present invention, are described in detail hereinafter.

1/Thus, according to one embodiment of the invention, the aqueous dispersion of polymer particles is an aqueous dispersion of particles of polyester-polyurethane and/or of polyether-polyurethane, which is/are in particular anionic.

The anionic nature of the polyester-polyurethanes and of the polyether-polyurethanes used according to the invention is due to the presence, in their constitutive units, of groups comprising a carboxylic acid or sulphonic acid function.

The polyester-polyurethane or polyether-polyurethane particles used according to the invention are generally sold in the form of aqueous dispersions.

The particle content of said dispersions currently available on the market ranges from approximately 20% to approximately 50% by weight relative to the total weight of the dispersion.

Among the dispersions of anionic polyester-polyurethane that can be used in the aqueous varnishes according to the invention, mention may in particular be made of the one sold under the name “Avalure UR 405®”, by the company Noveon.

Among the dispersions of anionic polyether-polyurethane particles that can be used according to the invention, mention may in particular be made of those sold under the name “Avalure UR 450®” by the company Noveon, and under the name “Neorez R 970®” by the company DSM.

According to a specific embodiment of the invention, use may be made of a mixture of commercially available dispersions consisting of anionic polyester-polyurethane particles as defined above and of anionic polyether-polyurethane particles also defined above.

For example, use may be made of a mixture consisting of the dispersion sold under the name “Sancure 861®” or a mixture of the one sold under the name “Avalure UR 405®” and of the one sold under the name “Avalure UR 450®”, these dispersions being sold by the company Noveon.

2/According to one variant of the invention, the aqueous dispersion of polymer particles is an aqueous dispersion of acrylic polymer.

In the context of the present invention, the term “acrylic polymer” is intended to mean polymers comprising at least 50% at least of monomer units chosen from acrylic acid, methacrylic acid and their esters, in particular from C₁-C₁₈ alkyl(meth)acrylate monomers as reported hereinafter.

The acrylic polymers may comprise other monomers, in particular derived from ethylenically unsaturated monomers. In this respect, styrene monomers or styrene-derived monomers may also be included in such an acrylic polymer.

Thus, according to one embodiment, the acrylic polymer may be a styrene/acrylate copolymer, and in particular a polymer chosen from copolymers derived from the polymerization of at least one styrene monomer and at least one C₁-C₁₈ alkyl(meth)acrylate monomer.

As styrene monomer that can be used in the invention, mention may, for example, be made of styrene or alpha-methylstyrene, and in particular styrene.

The C₁-C₁₈ alkyl(meth)acrylate monomer is in particular a C₁-C₁₂ alkyl(meth)acrylate, and more particularly a C₁-C₁₀ alkyl(meth)acrylate. The C₁-C₁₈ alkyl(meth)acrylate monomer may be chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl(meth)acrylate and stearyl(meth)acrylate.

Acrylic polymers in an aqueous dispersion that may be used according to the invention include the styrene-acrylate copolymer sold under the name “Joncryl SCX-8211®” by the company BASF, the acrylic polymer sold under the reference “Acronal® DS-6250” by the company BASF and the acrylic copolymer “Joncryl® 95” sold by the company BASF.

According to a specific embodiment of the invention, the aqueous dispersion of polymer particles is an aqueous dispersion of acrylic polymer.

In the context of the present invention, the term “acrylic polymer” is intended to mean polymers comprising at least 50% at least of monomer units chosen from acrylic acid, methacrylic acid and their esters.

3/According to another specific embodiment of the invention, the aqueous dispersion used comprises a blend of at least two film-forming polymers in the form of solid particles, that differ by virtue of their respective glass transition temperatures (Tg).

In particular, according to one embodiment of the invention, the composition in accordance with the invention may comprise at least a first film-forming polymer in the dispersed state and at least a second film-forming polymer in the dispersed state, said first and second polymers having different Tgs, and the Tg of the first polymer (Tg1) is preferably higher than the Tg of the second polymer (Tg2). In particular, one of the two polymers may have a dispersing function. In particular, the difference between Tg1 and Tg2 is, in absolute value, at least 10° C., preferably at least 20° C.

More specifically, the composition comprises, in an acceptable aqueous medium:

a) solid particles, dispersed in the aqueous medium, of a first film-forming polymer having at least one glass transition temperature Tg1 greater than or equal to 20° C., it being possible for said polymer to also have the dispersing function, and

b) solid particles, dispersed in the aqueous medium, of a second film-forming polymer having at least one glass transition temperature Tg2 of less than or equal to 70° C.

This dispersion generally results from a mixture of two aqueous dispersions of film-forming polymer.

The first film-forming polymer has at least one, especially has a glass transition temperature Tg1 of greater than or equal to 20° C., especially ranging from 20° C. to 150° C., and advantageously greater than or equal to 40° C., especially ranging from 40° C. to 150° C., and in particular greater than or equal to 50° C., especially ranging from 50° C. to 150° C.

The second film-forming polymer has at least one, especially has a glass transition temperature Tg2 of less than or equal to 70° C., especially ranging from −120° C. to 70° C., and in particular less than 50° C., especially ranging from −60° C. to +50° C., and more particularly ranging from −30° C. to 30° C.

The glass transition temperature (Tg) of a polymer is measured by DMTA (dynamic and mechanical temperature analysis) as described below.

To measure the glass transition temperature (Tg) of a polymer, viscoelasticimetry tests are performed with a DMTA machine from “Polymer Laboratories”, on a sample of film. This film is prepared by casting the aqueous dispersion of film-forming polymer in a teflon-coated matrix and then drying at 120° C. for 24 hours. A film is thus obtained, from which the test pieces are cut out (for example using a hole punch). These test pieces are typically approximately 150 μm thick, from 5 to 10 mm wide and have useful length of approximately 10 to 15 mm. This sample is subjected to a tensile stress. The sample undergoes a static force of 0.01 N, on which is superimposed a sinusoidal displacement of 8 μm at a frequency of 1 Hz. The test is thus performed in the linear range, at low levels of bending. This tensile stress is applied to the sample at temperatures ranging from −150° C. to +200° C., with a temperature variation of 3° C. per minute.

The complex modulus E*=E′+iE″ of the polymer tested is then measured as a function of the temperature.

From these measurements, the dynamic moduli E′, E″ and the damping power: tgδ=E″/E′ are deduced.

Next, the curve of the tgδ values as a function of the temperature is plotted; this curve exhibits at least one peak. The glass transition temperature Tg of the polymer corresponds to the temperature at which the top of this peak lies.

When the curve has at least two peaks (in this case, the polymer has at least 2 Tgs), the Tg value of the polymer tested is taken as the temperature for which the curve exhibits a peak of highest amplitude (i.e. corresponding to the largest value of tgδ; in this case, only the “predominant” Tg is considered as the Tg value of the polymer tested).

In the present invention, the transition temperature Tg1 corresponds to the “predominant” Tg (within the meaning defined above) of the first film-forming polymer when the latter has at least 2 Tgs; the glass transition temperature Tg2 corresponds to the “predominant” Tg of the second film-forming polymer when the latter has at least 2 Tgs.

The first film-forming polymer and the second film-forming polymer may be chosen, independently of one another, from radical polymers, polycondensates and polymers of natural origin, as defined above, having the glass transition temperature characteristics defined above.

As first film-forming polymer in an aqueous dispersion, use may be made of the aqueous dispersions of polymer sold under the names “NeoRez R-989®” by the company DSM, and “Joncryl 95” and “Joncryl® 8211” by the company BASF.

As second film-forming polymer in an aqueous dispersion, use may, for example, be made of the aqueous dispersions of polymer sold under the names “Avalure® UR-405” and “Avalure UR-460” by the company Noveon, “Acrilem IC89RT®” by the company ICAP or “Neocryl A-45” by the company DSM.

The film-forming polymer of the aqueous dispersion “Avalure® UR-460” is a polyurethane obtained by polycondensation of poly(tetramethylene oxide), of tetramethylxylylene diisocyanate, of isophorone diisocyanate and of dimethylolpropionic acid.

According to a most particularly preferred embodiment of the invention, the combination of styrene/acrylate polymer dispersion such as the dispersion sold under the reference “Joncryl 8211®” by BASF and of acrylic polymer dispersion such as the dispersion sold under the reference “Neocryl A-45®” by DSM is used as first and second film-forming polymers in an aqueous dispersion.

According to another preferred embodiment of this specific embodiment of point 3/above of the invention, a dispersion of acrylic polymer such as the dispersion sold under the reference “Joncryl 95®” by BASF is used as first film-forming polymer in an aqueous dispersion and a dispersion of anionic polyurethane polymer sold under the reference “Avalure UR405®” by DSM is used as second film-forming polymer.

In this embodiment of point 3/above, the total content with respect to solids of the first film-forming polymer and of the second film-forming polymer is generally from 0.1% to 60% by weight, relative to the total weight of the composition, in particular from 1% to 50%, and more particularly from 5% to 40% by weight.

4/According to yet another embodiment of the invention, the aqueous dispersion used comprises at least one specific multiphase aqueous dispersion.

More specifically, it comprises, in an acceptable aqueous medium, a dispersion of particles, the particles comprising at least one at least partially outer, flexible phase comprising at least one flexible polymer having at least one glass transition temperature Tg2 and at least one at least partially inner, rigid phase, the rigid phase being an amorphous material having at least one glass transition temperature Tg1, Tg1 being higher than Tg2, the flexible polymer being attached at least partially by chemical grafting to the rigid phase.

In particular, the difference between Tg1 and Tg2 is, in absolute value, at least 10° C., preferably at least 20° C.

The particles in accordance with the invention, also referred to as multiphase particles (or composites), are particles comprising at least one flexible phase and at least one rigid phase.

The flexible polymer of the particles in dispersion may have at least one glass transition temperature of less than or equal to 70° C., especially ranging from −120° C. to 70° C., and more particularly less than or equal to 30° C., especially ranging from −60° C. to 30° C.

The flexible polymer may be chosen from block and/or random polymers. The expression “block and/or random polymers” is intended to mean polymers in which the distribution of the monomers on the main chain or the pendent links is in blocks and/or random.

The flexible polymer may be chosen from radical polymers, polycondensates and silicone polymers. The flexible polymer may be chosen from polyacrylics, polymethacrylics, polyamides, polyurethanes, polyolefins, in particular polyisoprenes, polybutadienes or polyisobutylenes (PIBs), polyesters, polyvinyl ethers, polyvinyl thioethers, polyoxides, polysiloxanes, and in particular polydimethylsiloxanes (PDMSs), and combinations thereof. The term “combinations” is intended to mean the copolymers that can be formed from the monomers that result in the formation of said polymers.

In particular, the flexible polymer may be chosen from poly(meth)acrylics, polyurethanes, polyolefins and polysiloxanes.

The amorphous material of the rigid phase has a glass transition temperature of greater than 20° C., especially ranging from 20° C. to 150° C., in particular greater than or equal to 30° C., especially ranging from 30° C. to 150° C., preferably greater than or equal to 40° C., more particularly ranging from 40° C. to 150° C., or even greater than or equal to 50° C., especially ranging from 50° C. to 150° C.

The amorphous material of the rigid phase may be a polymer, in particular a block and/or random polymer. It may be a polymer chosen from polyacrylics, polymethacrylics, for instance poly((meth)acrylic acid)s, poly(meth)acrylamides, polyvinyls, polyvinyl esters, polyolefins, polystyrenes, polyvinyl halides such as polyvinyl chloride (PVC), polyvinylnitriles, polyurethanes, polyesters, polyamides, polycarbonates, polysulphones, polysulphonamides, polycyclics having a carbon-based ring in the main chain, such as polyphenylenes, polyoxyphenylenes, and combinations thereof.

Advantageously, the amorphous material of the rigid phase may be a polymer chosen from polyacrylics, polymethacrylics, for instance poly((meth)acrylic acid)s, poly(meth)acrylamides, polyvinyls, polyvinyl esters, polyolefins, polystyrenes, polyvinyl halides such as polyvinyl chloride (PVC), polyvinyl nitriles, polyurethanes, polyamides and polyesters.

According to a specific embodiment of the invention, the flexible and rigid phases of the multiphase particles may comprise at least one radical polymer obtained by, or essentially by, polymerization of monomers chosen from the group formed by:
(meth)acrylic acid esters, such as alkyl(meth)-acrylates, in particular having a C₁-C₈ alkyl group,
linear or branched carboxylic acid vinyl esters, such as vinyl acetate or vinyl stearate,
styrene and its derivatives, such as chloro-methylstyrene, alpha-methylstyrene,
conjugated dienes, such as butadiene or isoprene,
acrylamide, methacrylamide and acrylonitrile,
vinyl chloride, and
(meth)acrylic acid.

The selection of monomers (nature and content), which may be a single monomer or a mixture of at least two monomers, of the flexible polymer and of the amorphous material of the rigid phase is determined by the glass transition temperature that it is desired to confer on each polymer.

The polymers of the rigid and/or flexible phases may be crosslinked with monomers having at least two copolymerizable double bonds, for example chosen from:
conjugated dienes, such as butadiene or isoprene,
alpha, beta-unsaturated carboxylic acid allyl esters, such as allyl acrylate or allyl methacrylate,
alpha, beta-unsaturated dicarboxylic acid allyl esters, such as diallyl maleate,
polyacrylics or polymethacrylics generally comprising at least two ethylenic unsaturations, such as ethylene glycol dimethacrylate, 1,3-butylene glycol dimethacrylate, 1,4-butanediol diacrylate or pentaneerythrityl tetraacrylate;
polyvinyls such as divinylbenzene or trivinylbenzene, and
polyallyls such as triallyl cyanurate.

Chemical grafting makes it possible, through the formation of covalent bonds, to stably link the rigid phase and the flexible phase of the multiphase particles.

The chemical grafting may be carried out by radical block polymerization (also referred to as sequential polymerization) according to the protocols well known to those skilled in the art. The block polymerization consists in polymerizing, in a first step, the monomers of the rigid polymer (polymer forming the rigid phase of the particles) and then continuing, in a second step, the polymerization with the monomers forming the flexible polymer (polymer forming the flexible phase of the particles). In this manner, the polymer chains of the flexible phase are linked at least in part by covalent bonding to the polymer chains of the rigid phase, the covalent bonding resulting from the polymerization of a monomer of the flexible polymer with a monomer of the rigid polymer. Advantageously, the monomers of the polymer of the outer flexible phase have a greater affinity for the dispersing medium than the monomers of the polymer of the inner rigid phase.

The flexible polymer may be grafted onto the rigid polymer by means of a grafting monomer, it being possible for the latter to be a monomer having several double bonds (ethylenic bonds), in particular a monomer having two ethylenic double bonds. The grafting monomer may be a conjugated diene such as those described above or an alpha, beta-unsaturated dicarboxylic acid allyl ester (in particular diester) such as those described above (for instance, diallyl maleate) which have two polymerizable functions (ethylenic double bond) of different reactivities: one of the polymerizable functions (ethylenic double bond) of the grafting monomer is polymerized with the polymer of the amorphous material of the rigid phase (rigid polymer) and the other polymerizable function (ethylenic double bond) of the same grafting monomer is polymerized with the flexible polymer.

When the flexible polymer or the polymer of the rigid phase is a polycondensate, use is in particular made of a polycondensate having at least one ethylenic unsaturation capable of reacting with a monomer also comprising an ethylenic unsaturation so as to form a covalent bond with the polycondensate. Polycondensates comprising one or more ethylenic unsaturations are in particular obtained by polycondensation of monomers such as allyl alcohol, vinylamine or fumaric acid. It is, for example, possible to polymerize vinyl monomers with a polyurethane having vinyl groups in or at the end of the polyurethane chain and thus to graft a vinyl polymer onto a polyurethane; a dispersion of particles of such a grafted polymer is in particular described in the publications “The structure and properties of acrylic-polyurethane hybrid emulsions”, Hiroze M., Progress in Organic Coatings, 38 (2000), pages 27-34; “Survey of the applications, properties, and technology of crosslinking emulsions”, Bufkin B, Journal of Coatings technology, vol. 50, No. 647, December 1978.

The same grafting principle applies for silicones, using silicones comprising vinyl groups which make it possible to polymerize vinyl monomers onto the silicone and thus to graft vinyl polymer chains onto a silicone.

The particles comprising rigid and flexible phases generally have a size ranging from 1 nm to 1 μm, in particular ranging from 10 nm to 1 μm.

The flexible phase may be present in the particles at a content of at least 1% by volume, relative to the total volume of the particle, especially of at least 5% by volume, in particular of at least 10% by volume, and more particularly of at least 25% by volume. The flexible phase may be present in the particles at a content ranging up to 99.999% by volume, especially up to 99.9% by volume, in particular up to 99% by volume, and more particularly up to 95% by volume. In particular, the flexible phase may be present in the particles at a content ranging from 1% to 99.999% by volume, in particular ranging from 5% to 99.9% by volume, and more particularly ranging from 10% to 99.9% by volume, especially ranging from 25% to 99.9% by volume, and more particularly ranging from 50% to 95% by volume, or even ranging from 50% to 90% by volume.

In any event, the rigid phase and the flexible phase are incompatible, i.e. they can be distinguished using techniques well known to those skilled in the art, such as, for example, the technique of observation by electron microscopy or the measurement of the glass transition temperatures of the particles by differential calorimetry. The multiphase particles are therefore nonhomogeneous particles.

The morphology of the flexible and rigid phases of the dispersed particles may, for example, be of core-shell type, with parts of shell completely surrounding the core, but also core-shell type with a multiplicity of cores, or an interpenetrating network of phases. In the multiphase particles, the flexible phase is at least partially, in particular predominantly, external, and the rigid phase is at least partially, in particular predominantly, internal.

The multiphase particles may be prepared by consecutive polymerization series, with various types of monomers. The particles of a first group of monomers are generally prepared in a separate step, or formed in situ by polymerization. Subsequently, or at the same time, at least one other group of other monomers is polymerized during at least one additional polymerization step. The particles thus formed have at least one structure that is at least partially internal, or as a core, and at least one structure that is at least partially external, or as a shell. The formation of a “multilayer” heterogeneous structure is thus possible. This can result in a large variety of morphologies, of the core-shell type, but also, for example, with fragmented inclusions of the rigid phase in the flexible phase. According to the invention, it is essential for the structure in the flexible phase that is at least partially external to be more flexible than the structure in the rigid phase that is at least partially internal.

According to a specific embodiment of the invention, the multiphase particles may be dispersed in an aqueous medium, in particular a hydrophilic medium. The aqueous medium may consist predominantly of water, and in particular almost completely of water. These dispersed particles thus form an aqueous dispersion of polymer, generally known as latex or pseudolatex. The term “latex” is intended to mean an aqueous dispersion of polymer particles such that it can be obtained by emulsion polymerization of at least one monomer.

The dispersion of multiphase particles is generally prepared by at least one essentially aqueous continuous-phase emulsion polymerization, using reaction initiators, such as photochemical initiators or thermal initiators for radical polymerization, optionally in the presence of additives such as stabilizers, chain-transfer agents and/or catalysts.

According to a specific embodiment of the invention, the film-forming polymer is an aqueous dispersion of particles of polyester-polyurethane and/or of polyether-polyurethane, which is/are in particular anionic, or an aqueous dispersion of acrylic polymer particles.

Aqueous dispersions of film-forming polymer that may be used include the acrylic dispersions sold under the names “Acronal DS-6250®” by the company BASF, “Neocryl A-45®”, “Neocryl XK-90®”, “Neocryl A-1070®”, “Neocryl A-1090®”, “Neocryl BT-62®”, “Neocryl A-1079®” and “Neocryl A-523®” by the company DSM, “Joncryl 95®” and “Joncryl 8211®” by the company BASF, “Daitosol 5000 AD®” or “Daitosol 5000 SJ” by the company Daito Kasey Kogyo; “Syntran 5760” by the company Interpolymer, or else the aqueous dispersions of polyurethane sold under the names “Neorez R-981®” and “Neorez R-974®” by the company DSM, “Avalure UR-405®, “Avalure UR-410®”, “Avalure UR-425®”, “Avalure UR-450®”, “Sancure 875®”, “Avalure UR 445®” and “Avalure UR 450®” by the company Noveon, “Impranil 85®” by the company Bayer, the sulphopolyesters sold under the trade mark “Eastman AQ®” by the company Eastman Chemical Products, vinyl dispersions such as “Mexomere PAM”, aqueous dispersions of polyvinyl acetate such as “Vinybran®” from the company Nisshin Chemical or those sold by the company Union Carbide, aqueous dispersions of vinylpyrrolidone/dimethylaminopropylmethacrylamide/lauryldimethylpropylmethacrylamidoammonium chloride terpolymer, such as “Styleze W®” from ISP, aqueous dispersions of polyurethane/polyacrylic hybrid polymers, such as those sold under the reference “Hybridur®” by the company Air Products or “Duromere®” by National Starch, dispersions of particles of core-shell type, such as those sold by the company Arkema under the reference “Kynar®” (core: fluorinated-shell: acrylic) or else those described in U.S. Pat. No. 5,188,899 (core: silica-shell: silicone), and mixtures thereof.

The aqueous dispersions of particles used in the invention may also comprise various additives. They may in particular comprise an auxiliary film-forming agent that promotes the formation of a film with the particles of film-forming polymers. Such an auxiliary film-forming agent may be chosen from all the compounds known to those skilled in the art to be capable of performing the desired function, and in particular may be chosen, from plasticizers and coalescence agents.

When the aqueous dispersion of particles is obtained according to the third variant of the invention, i.e. when the aqueous dispersion used comprises a blend of at least two film-forming polymers, in the form of solid particles, differing by virtue of their respective glass transition temperatures Tg (Tg1 being higher than Tg2, in particular the difference between Tg1 and Tg2 being greater than or equal to 10° C. in absolute value, and Tg1 preferably being greater than or equal to 20° C. and Tg2 preferably being less than or equal to 70° C.), the dispersion may be free or substantially free of plasticizer.

According to a specific embodiment of the invention, the composition is a nail varnish composition with an aqueous continuous phase, comprising a particulate phase having an average particle size of less than or equal to 0.5 μm, at least one film-forming polymer in the form of an aqueous dispersion of particles chosen from acrylic dispersions, aqueous dispersions of polyurethane, sulphopolyesters, vinyl dispersions, aqueous dispersions of polyvinyl acetate, aqueous dispersions of vinylpyrrolidone/dimethylaminopropylmethacrylamide/lauryldimethylpropylmethacrylamidoammonium chloride terpolymer, aqueous dispersions of polyurethane/polyacrylic hybrid polymers, dispersions of particles of core-shell type, and mixtures thereof, and a dispersing agent chosen from the ionic polymers as described above.

According to another specific embodiment of the invention, the composition is a nail varnish composition with an aqueous continuous phase, comprising a particulate phase having an average particle size of less than or equal to 0.5 μm, at least one film-forming polymer in the form of an aqueous dispersion of particles of polyester-polyurethane and/or of polyether-polyurethane, which is/are in particular anionic, or of an aqueous dispersion of acrylic polymer particles, and a dispersing agent chosen from a polyacrylic acid in the form of a salt and a styrene/acrylic acid copolymer in the form of a salt.

Other Additives

All the film-forming polymers mentioned above may be combined with at least one auxiliary film-forming agent.

The auxiliary film-forming agent may be chosen from all the compounds known to those skilled in the art to be capable of performing the desired function, and may in particular be chosen from plasticizers and coalescence agents for the film-forming polymer.

In particular, mention may be made, alone or as a mixture, of the usual plasticizers or coalescence agents, such as:
glycols and derivatives thereof, such as diethylene glycol ethyl ether, diethylene glycol methyl ether, diethylene glycol butyl ether or alternatively diethylene glycol hexyl ether, ethylene glycol ethyl ether, ethylene glycol butyl ether or ethylene glycol hexyl ether;
glycol esters,
propylene glycol derivatives, and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,
esters of acids, in particular carboxylic acids, such as citrates, in particular triethyl citrate, tributyl citrate, triethyl acetyl citrate, tributyl acetyl citrate, 2-triethylhexyl acetyl citrate; phthalates, in particular diethyl phthalate, dibutyl phthalate, dioctyl phthalate, dipentyl phthalate, dimethoxyethyl phthalate, 2-ethylhexyl butyl phthalate; phosphates, in particular tricresyl phosphate, tributyl phosphate, triphenyl phosphate, tributoxyethyl phosphate; tartrates, in particular dibutyl tartrate; adipates such as diisobutyl adipate or diethyl adipate; carbonates; sebacates such as dimethyl sebacate or dibutyl sebacate; ethyl stearate, 2-ethylhexyl palmitate, the ester of tert-butylic acid and of 2,2,4-trimethyl-1,3-pentanediol, benzyl benzoate, butyl acetylricinoleate, glyceryl acetylricinoleate, butyl glycolate, camphor, glyceryl triacetate and N-ethyl-o,p-toluenesulphonamide,
oxyethylenated derivatives, such as oxyethylenated oils, in particular plant oils such as castor oil; silicone oils,
mixtures thereof.

According to a preferred embodiment, the plasticizer is chosen from diisobutyl adipate, the ester of tert-butylic acid and of 2,2,4-trimethyl-1,3-pentanediol, diethyl adipate, diethyl phthalate, dibutyl phthalate, dioctyl phthalate, 2-ethylhexyl butyl phthalate, dimethyl sebacate, dibutyl sebacate, ethyl stearate, 2-ethylhexyl palmitate, dipropylene glycol butyl ether, and mixtures thereof.

The type and the amount of plasticizer and/or of coalescence agent may be chosen by those skilled in the art on the basis of their general knowledge.

For example, the content of plasticizer and/or of coalescence agent may range from 0.1% to 20%, especially from 0.5% to 10%, and in particular from 1% to 5% by weight relative to the total weight of the composition.

The composition in accordance with the present invention may also comprise water-soluble or liposoluble dyes at a content ranging from 0.01% to 10% by weight, especially ranging from 0.01% to 5% by weight, relative to the total weight of the resulting film. The liposoluble dyes are, for example, Sudan red, DC Red 17, DC Green 6, β-carotene, soybean oil, Sudan brown, DC Yellow 11, DC Violet 2, DC Orange 5 and quinoline yellow. The water-soluble dyes are, for example, beetroot juice and methylene blue.

The composition in accordance with the invention may also contain ingredients commonly used in cosmetics, and more especially in the cosmetic and/or nail care field. They may in particular be chosen from vitamins, trace elements, softeners, sequestering agents, basifying or acidifying agents, spreading agents, wetting agents, thickeners, dispersing agents, preserving agents, UV-screens, active agents, moisturizers, fragrances, neutralizing agents, stabilizers, antioxidants and mixtures thereof.

Mention may also be made of antifoams, for instance polydimethylsiloxanes, in particular having a viscosity ranging from 200 to 500 cSt, for instance the product DC200 Fluid sold by Dow Corning (350 cSt).

Thus, when the compositions in accordance with the invention are more particularly intended for the care of natural nails, they may in particular incorporate, by way of active agents, hardeners for keratin materials, active agents that act on nail growth, for instance methylsulphonylmethane, and/or active agents for treating various conditions located in the nail, for instance onychomycosis.

The amounts of these various ingredients are those conventionally used in this field, and are, for example, from 0.01% to 20%, and in particular from 0.01% to 10% by weight, relative to the total weight of the composition in accordance with the invention.

Felt-Tip Applicator

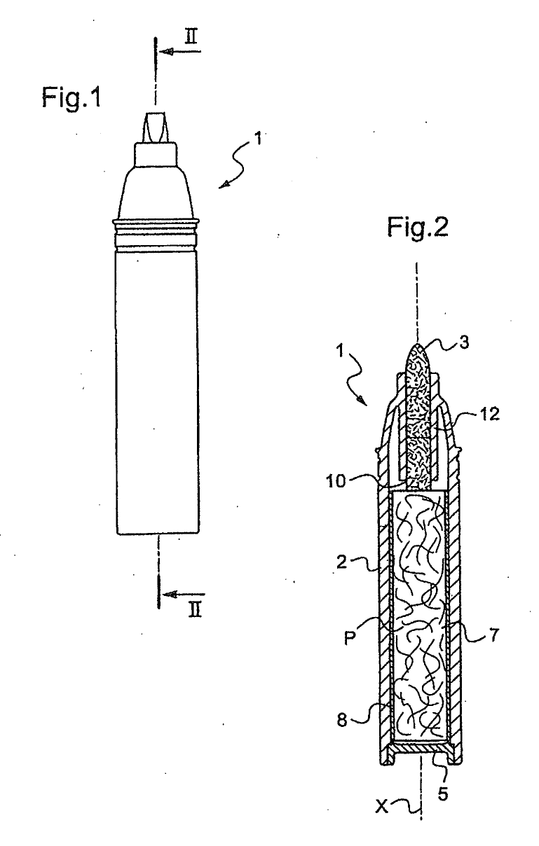

FIG. 1 represents a schematic elevation view of an example of an applicator pen suitable for the application of the composition.

FIG. 2, which is a longitudinal section along II-II of FIG. 1, comprises a body 2 of elongated shape along a longitudinal axis X, equipped, at one end, with an application element 3.

The applicator 1 also comprises a closure element which, in the example illustrated, is in the form of a cap that can attach to the body 2, for example by click-fastening in order to obtain leaktight closure of the applicator when it is not in use.

In the example considered, the body 2 is closed at the end opposite the application element 3 by a base 5 which is, for example, click-fastened or held by friction in the body 2.

The applicator 2 contains a product P which impregnates, for example, a block 7 of a porous material, for example cotton wool, which can be surrounded by a sheath 8, the outer diameter of which corresponds substantially to the inner diameter of the body 2.

In the example illustrated, the block 7 takes up most of the height of the applicator 1 and, at one end, comes up against the base 5.

The product is transported to the application element 3 by capillary action. In the example illustrated, a wick 10 is applied, at one end, against the block 7 and, at the other end, against the application element 3. The wick 10, and also the application element 3, are fitted into a shaft 12 which, in the example considered, is made as a single piece with the body 2 by moulding a thermoplastic.

The application element 3 also forms a wick and may or may not be made of the same material as the wick 10.

The application element 3, and also the wick 10, may be made, independently of one another, of any materials that make it possible to transport the product by capillary action (subsequently referred to as “porous materials”), in particular any material made of compressed fibres, of porous composite, of foam, of cellulose, of mineral or plastic frit, for example of elastomer bead frit, etc.

The wall of the body may be substantially non-deformable, the transfer of the product onto the region to be treated being carried out by capillary action without the creation of any increased pressure in the reservoir. The application element 3 is axially fixed relative to the body 2 during the application, in the example considered.

Of course, the invention is not limited to the implementation example described, and many modifications may be introduced without departing from the context of the present invention.

According to one embodiment, the wick is directly in contact with the block of porous material.

According to another specific embodiment, no fraction of the composition remains free in the applicator containing the composition. In other words, according to this embodiment, the block 7 is impregnated with the entire composition.

For example, the application element 3 may come directly into contact with the block 7 in the reservoir defined by the body 2 without an intermediate wick 10.

In a further variant, the block 7 is absent.

The application element 3 may be given various shapes. In the example illustrated, the tip of the application element 3 is wedge-shaped, when observed from the side in the direction of the arrows II of FIG. 1, but the tip of the application element could be given other shapes, for example conical, frustoconical, dual-tip, cut-off sides, concave edges, or the like. The application element 3 may also be covered with fibres over its surface, for example by flocking.

The wall of the body 2 could, where appropriate, be made of a more flexible material so as to allow the user to create an increased pressure inside the reservoir in order to increase the flow rate of product upon application, for example.

In a further variant, the application element 3 is made monolithic with the block 7.

Preferably, in the context of the present invention, the application element 3 remains in permanent fluidic communication with the composition contained in the reservoir.

More preferably, the applicator in accordance with the present invention is devoid of a valve, in particular between the reservoir and the application element 3.

Finally, the applicator in accordance with the present invention preferably does not require any pressure on the body 2 in order to promote the flow of the composition through the application element 3.

In order to use the applicator, the user holds the body 2 like a pen and can bring the tip of the application element into contact with the nail, in particular from the inner edge to the outer edge of the nail.

A subject of the invention is also an assembly as defined above, in which the felt-tip applicator is an applicator pen comprising a block of porous material impregnated with the composition, and a wick for transporting the composition by capillary action. Such an applicator allows good application of the composition onto the nails. In fact, firstly, the composition can be applied irrespective of the position of the applicator: the makeup can therefore be applied without the applicator having to be systematically held vertically, and without it running. Moreover, such an assembly makes it possible to deposit several successive layers, without degrading the layer(s) already deposited, and without detaching them. A subject of the invention is also a method for making up the nails comprising at least one step of application of a composition with a continuous aqueous phase, comprising a particulate phase having an average particle size of less than or equal to 1 μm, at least two film-forming polymers and a dispersing agent, using a felt-tip applicator.

Finally, a subject of the invention is a method for making up and/or for the non-therapeutic care of keratin materials chosen from the skin, the lips and the eyelashes, and the teeth, comprising at least one step of application to said keratin materials or to the teeth of a composition with a continuous aqueous phase, comprising a particulate phase having an average particle size of less than or equal to 1 μm, at least one film-forming polymer and a dispersing agent, using a felt-tip applicator.

The examples which follow illustrate the present invention. The contents are indicated as percentages by weight.

### EXAMPLES

### Examples 1 to 4

### Nail Varnish

Compositions without Plasticizer

| | | Example 1 | Example 2 |
|---|---|---|---|
| Trade name | Ingredient | % | % |
| Mixture of 15% of | Pigmentary paste | 20 | 20 |
| DC Red 7 pigments | | | |
| and 10% of styrene/ | | | |
| acrylic copolymer | | | |
| dispersing agent in | | | |
| the form of a salt | | | |
| in water | | | |
| | Water | 30 | 30 |
| Joncryl 95⁽²⁾ | Acrylic latex, 30% | 37.5 | |
| | with respect to | | |
| | solids of polymer | | |
| Avalure UR405⁽³⁾ | Polyurethane latex, | 12.5 | |
| | 35% with respect to | | |
| | solids of polymer | | |
| Joncryl 8211⁽⁴⁾ | Hard acrylic latex, | | 30 |
| | 44% with respect to | | |
| | solids of polymer | | |
| | (Tg = 60° C.) | | |
| Neocryl A-45⁽⁵⁾ | Soft acrylic latex, | | 20 |
| | 37.5% with respect | | |
| | to solids of | | |
| | polymer (Tg = 15° C.) | | |

⁽²⁾sold by the company BASF
⁽³⁾sold by the company Noveon
⁽⁴⁾sold by the company BASF
⁽⁵⁾sold by the company DSM

The products of Example 1 or 2, after application with the pen, result in the formation of a uniform film without defects on the nails. The makeup result is smooth and shiny. During successive applications, the prior layers are preserved, i.e. they are neither detached nor degraded.

Compositions with Plasticizer

| | Example 3 | Example 4 |
|---|---|---|
| Starting material | % | % |
| Mixture of 15% of DC Red 7 | 17 | 17 |
| pigments and of 10% of | | |
| styrene/acrylic copolymer | | |
| dispersing agent in the | | |
| form of a salt in water | | |
| Acronal DS-6250⁽¹⁾ | 60.3 | 48.3 |
| Diisobutyl adipate | 1.9 | 1.5 |
| Joncryl 8211⁽⁴⁾ | 20.8 | 33.2 |

The formulas presented in this table are film-forming and produce smooth and shiny films. They can be applied using a felt-type pen.

### Example 5

### Composition for Coating Teeth

Composition with Plasticizer

| | |
|---|---|
| Mixture of 15% of yellow iron oxides | 17 |
| and 10% of styrene/acrylic copolymer | |
| dispersing agent in water | |
| Acronal DS-6250⁽¹⁾ | 60.3 |
| Diisobutyl adipate | 1.9 |
| Joncryl 8211⁽⁴⁾ | 20.8 |

1. Assembly for the application of a nail varnish composition or a composition for making up and/or caring for keratin materials chosen from the skin, the lips and the eyelashes, or the teeth, comprising:
a felt-tip applicator, said applicator being an applicator pen comprising a block of porous material impregnated with the composition, and a wick for transporting the composition by capillary action, and
a nail varnish composition, or a composition for making up and/or caring for keratin materials, with a continuous aqueous phase comprising a particulate phase having an average particle size of less than or equal to 1 μm and at least two film-forming polymers.

2. Assembly for the application of a nail varnish composition or a composition for making up and/or caring for keratin materials chosen from the skin, the lips and the eyelashes, or the teeth, comprising:
a felt-tip applicator, said applicator being an applicator pen comprising a block of porous material impregnated with the composition, and a wick for transporting the composition by capillary action, and
a nail varnish composition, or a composition for making up and/or caring for keratin materials, with a continuous aqueous phase comprising a particulate phase having an average particle size of less than or equal to 0.5 μm, at least one film-forming polymer and a dispersing agent.

3. Assembly according to claim 1, in which the particulate phase comprises particulate colouring substances, pearlescent agents and/or fillers.

4. Assembly according to claim 3, in which the average size of the particles consisting of the particulate colouring substances, the pearlescent agents and/or the fillers is between 0.1 and 1 μm.

5. Assembly according to claim 1, in which the particulate phase comprises particles of pigment(s).

6. Assembly according to the preceding claim 5, in which the pigment(s) is (are) chosen from organic and mineral pigments.

7. Assembly according to claim 5, in which the pigment(s) is (are) organic pigment(s) chosen from carbon black, pigments of D & C type, and lakes based on cochineal carmine, on barium, on strontium, on calcium and on aluminium.

8. Assembly according to claim 5, in which the pigment(s) is (are) mineral pigment(s) chosen from titanium dioxide, optionally surface-treated, zirconium or cerium oxides, and also zinc oxides, iron oxides or chromium oxides, manganese violet, ultramarine blue, chromium hydrate and ferric blue, metal powders.

9. Assembly according to claim 1, in which the particulate phase is present in the composition at a content ranging from 0.01% to 20% by weight relative to the total weight of the composition.

10. Assembly according to claim 1, in which the dispersing agent is chosen from:
ionic surfactants,
nonionic surfactants;
ionic polymers;
nonionic polymers;
citric acid or alpha-hydroxy acids; and
mixtures thereof.

11. Assembly according to claim 1, in which the dispersing agent is chosen from anionic polymers.

12. Assembly according to claim 1, in which the dispersing agent is present at a content ranging from 0.01% to 10% by weight relative to the total weight of the composition.

13. Assembly according to claim 1, in which the film-forming polymer(s) is (are) in the dispersed state, chosen from acrylic dispersions, aqueous dispersions of polyurethane, sulphopolyesters, vinyl dispersions, aqueous dispersions of polyvinyl acetate, aqueous dispersions of vinylpyrrolidone/dimethylaminopropylmethacrylamide/lauryldimethylpropylmethacrylamidoammonium chloride terpolymer, aqueous dispersions of polyurethane/polyacrylic hybrid polymers, dispersions of particles of core-shell type, and mixtures thereof.

14. Assembly according to claim 1, in which the composition comprises at least a first film-forming polymer in the dispersed state and at least a second film-forming polymer in the dispersed state, said first and second polymers having different Tgs.

15. Assembly according to claim 14, in which the first film-forming polymer has a glass transition temperature Tg1 of greater than or equal to 20° C., and the second film-forming polymer has a glass transition temperature Tg2 of less than or equal to 70° C.

16. Assembly according to claim 1, in which the nail varnish composition or composition for making up and/or caring for keratin materials comprises from 0.1% to 60% by weight, solids of film-forming polymer, relative to the total weight of the composition.

17. Assembly according to claim 1, in which the composition also comprises a plasticizer.

18. Assembly according to claim 17, in which the plasticizer is chosen from diisobutyl adipate, the ester of tert-butylic acid and of 2,2,4-trimethyl-1,3-pentanediol, diethyl adipate, diethyl phthalate, dibutyl phthalate, dioctyl phthalate, butyl and 2-ethylhexyl phthalate, dimethyl sebacate, dibutyl sebacate, ethyl stearate, 2-ethylhexyl palmitate, dipropylene glycol butyl ether, and mixtures thereof.

19. Assembly according to claim 1, in which the composition also comprises water-soluble or liposoluble dyes.

20. Assembly according to claim 1, in which the composition has a viscosity ranging from 2 to 200 cps.

21. Method for making up the nails, or for making up and/or for the non-therapeutic care of keratin materials chosen from the skin, the lips and the eyelashes, or the teeth, comprising at least one step of application to said keratin materials or to the teeth of a composition with a continuous aqueous phase, comprising a particulate phase having an average particle size of less than or equal to 1 μm and at least two film-forming polymers, using a felt-tip applicator, said applicator being an applicator pen comprising a block of porous material impregnated with the composition, and a wick for transporting the composition by capillary action.

22. Method for making up the nails, or for making up and/or for the non-therapeutic care of keratin materials chosen from the skin, the lips and the eyelashes, or the teeth, comprising at least one step of application to said keratin materials or to the teeth of a composition with a continuous aqueous phase, comprising a particulate phase having an average particle size of less than or equal to 0.5 μm, at least one film-forming polymer and a dispersing agent, using a felt-tip applicator, said applicator being an applicator pen comprising a block of porous material impregnated with the composition, and a wick for transporting the composition by capillary action.
